(19) United States
(12) Patent Application Publication
(10) Pub. No.: US 2008/0286354 A1
(43) Pub. Date: Nov. 20, 2008

(54) COMPOSITION AND THERAPIES FOR HYPERLIPIDAEMIA-ASSOCIATED DISORDERS

(76) Inventor: THOMAS JULIUS BORODY, Five Dock (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/132,522

(22) Filed: Jun. 3, 2008

Related U.S. Application Data
(62) Division of application No. 10/257,371, filed on Jun. 13, 2003.

(30) Foreign Application Priority Data
Apr. 19, 2000 (AU) PQ6969
Oct. 19, 2000 (AU) PR0851

(51) Int. Cl. A61K 9/48; A61K 31/56; A61K 31/403; A61P 1/16

(52) U.S. Cl. 424/451; 514/171; 514/415

(57) ABSTRACT

The invention relates to pharmaceutical compositions comprising at least one fibrate and at least one bile acid, and to methods for the treatment of hyperlipidaemia or elevated liver function tests in a patient by administering at least one fibrate and at least one bile acid to the patient.

### TECHNICAL FIELD

The present invention relates to pharmaceutical compositions and methods for the treatment of hyperlipidaemia and elevated liver function tests.

### BACKGROUND TO THE INVENTION

Elevated concentrations of circulating lipid compounds in the blood such as cholesterol and triglycerides accompany a number of conditions. These include Type H diabetes, Primary Biliary Cirrhosis, Primary Sclerosing Cholangitis, various chronic hepatitis states (Hepatitis B and C), NASH (non-alcohol-induced steatohepatitis), and arterial disease including coronary artery disease, cerebro-vascular arterial disease, peripheral vascular disease, aortic aneurysms and carotid atherosclerotic conditions. Various lipid-lowering techniques have been used in the past to treat and to prevent the vascular events (such as cardiac failure, embolism, heart attacks and strokes) that is accompany hyperlipidaemic states. Such treatments have included dietary changes and control of high triglyceride levels circulating in the blood. The latter have been treated generally pharmacologically and lately with various ‘statins’. Included in the therapeutic agents used for treatment of hyperlipidaemia are various fibric acid derivatives. Some older fibric acid derivatives including clofibrate have had a passing place in the treatment of hyperlipidaemia but more recently new fibrates including fenofibrate, gemfibrozil, ciprofibrate, and even more recently fibrates containing piperidine, 4-hydroxypiperidine, piperidin-3-ene, and piperazine have joined the ranks of anti-lipid therapies. Of the newer molecules 2-[3-[1-(4-fluorobenzoyl)piperidin-4-yl]phenoxy-2-methylpropanoic acid may have the most promising properties. Bezafibrate (2-[4-[2-[(4-chlorobenzoyl)amino]-ethyl]phenoxy]-2-methylpropanoic acid) possesses activity as a therapeutic agent to reduce both cholesterol and triglycerides. However, in some situations a fibric acid derivative alone is inadequate in controlling the severe level of hyperlipidaemia that is present in many patients.

Ursodeoxycholic acid (UDCA) is a proven agent for the lowering of elevated liver function tests in Primary Biliary Cirrhosis (PBC) but has only minor effects upon hyperlipidaemia.

Accordingly, there is a need for an improved therapy for the treatment of hyperlipidaemia. There is also a need for an improved treatment of elevated liver function tests, especially when associated with conditions other than primary biliary cirrhosis.

### SUMMARY OF THE INVENTION

According to a first embodiment, the invention provides a pharmaceutical composition for the treatment of hyperlipidaemia, comprising at least one bile acid and at least one fibrate, together with one or more pharmaceutically acceptable carriers, diluents, adjuvants or excipients, with the proviso that if said bile acid is ursodeoxycholic acid, then said fibrate is other than bezafibrate.

According to a second embodiment, the invention provides a method of treating hyperlipidaemia in a patient in need of said treatment, comprising administering to said patient an effective amount of at least one bile acid and an effective amount of at least one fibrate.

According to a third embodiment, the invention provides a method of lowering elevated liver function tests in a patient in need of said treatment, comprising administering to said patient an effective amount of at least one bile acid and an effective amount of at least one fibrate, with the proviso that said elevated liver notion tests are not associated with primary biliary cirrhosis.

According to a fourth embodiment, the invention provides a method for the treatment of primary biliary cirrhosis in a patient in need of said treatment, comprising administering to said patient an effective amount of a fibrate and an effective amount of ursodiol bicarbonate or ursodiol sulfate, or a mixture thereof.

As used herein, the term “ursodiol bicarbonate” refers to a composition comprising ursodeoxycholic acid and sodium bicarbonate, preferably in an amount of from about 0.5 to 3 molar equivalents based on the amount of ursodeoxycholic acid. Such compositions are disclosed in U.S. Pat. No. 5,380,533. As used herein, the term “ursodiol sulfate” refers to the 3-sulfate, 7-sulfate or 3,7-disulfate of ursodeoxycholic acid or mixtures of any two or more thereof, which are disclosed in U.S. Pat. No. 5,763,435, The disclosures of U.S. Pat. Nos. 5,380,533 and 5,763,435 are incorporated herein by reference.

According to a fifth embodiment, the invention provides the use of an effective amount of at least one bile acid and an effective amount of at least one fibrate for the manufacture of a medicament for treating hyperlipidaemia in a patient in need of said treatment.

According to a sixth embodiment, the invention provides the use of an effective amount of at least one bile acid and an effective amount of at least one fibrate for the manufacture of a medicament for treating elevated liver function tests in a patient in need of said treatment, with the proviso that said elevated liver function tests are not associated with Primary Biliary Cirrhosis.

According to a seventh embodiment, the invention provides the use of an effective amount of at least one bile acid and an effective amount of at least one fibrate and an effective amount of at least one statin for the manufacture of a medicament for treating hyperlipidaemia in a patient in need of said treatment.

According to an eighth embodiment, the invention provides the use of an effective amount of ursodiol bicarbonate or ursodiol sulfate, or a mixture thereof, together with an effective amount of a fibrate for the manufacture of a medicament for the treatment of primary biliary cirrhosis.

### DETAILED DESCRIPTION OF THE INVENTION

The present inventor has noted a profound and surprising synergistic activity of a bile acid administered together with a fibrate in lowering lipids (both cholesterol and triglycerides) such as in patients whose elevated lipid levels are resistant to therapy in PBC. Triglyceride fall of >70% can be achieved using such a combination, especially when the fibrate is administered as a slow release formulation. This combination has also been found by the inventor to effectively lower liver function tests, including in PBC patients who were on a full dose of UDCA and had failed to improve their liver function tests. Hence, the combination of a bile acid and a fibrate can effectively treat this ‘Resistant PBC’ which fails to respond to the administration of UDCA alone,

In particular the present inventor has found that in PBC the combination of a bile acid and a fibrate enhances the lipid lowering effect of the fibrate in the PBC-associated hyperlipidaemia state, and when combined there is also a lowering of the markedly elevated liver Action tests. It is speculated by the present inventor that the lowering of elevated liver function tests may be etiologically related to the lipid lowering therapy. Hence, these two abnormalities may be jointly treatable and perhaps causally related.

The present invention thus derives from the unexpected synergy found when one or more bile acids is administered with one or more fibrates, for the treatment of hyperlipidaemia and for lowering elevated liver function tests. In one application, the compositions and methods of the present invention may be used to treat hyperlipidaemia together with elevated liver function tests in primary biliary cirrhosis. The invention also provides methods and compositions for the treatment of elevated liver faction tests in conditions other than those associated with PBC.

As used herein, the terra “fibrate” refers to pharmaceutically active derivatives of 2-phenoxy-2-methylpropanoic acid. Such derivatives are disclosed, for example, in U.S. Pat. Nos. 3,781,328, 3,948,973, 3,869,477, 3,716,583, 3,262,580, 3,723,446, 4,058,552, 3,674,836, 3,369,025, 3,984,413 and 3971798, Belgian patent no. 884722, United Kingdom patent no, 860303 and European patent application no. 607536, the disclosures of which are incorporated herein by reference.

Of the fibrates, bezafibrate alone has been shown to have only a modest effect on lowering alkaline phosphatase (Day et al, Metabolism 42 839 (1993)) but when combined with ursodeoxycholic acid has been shown to lower elevated liver Action tests associated with primary biliary cirrhosis (Nakai et al, Am. J. Gastroenterol. 95 326 (2000); Kurihara et al, Am. J. Gastroenterol. 95 2990 (2000); and Miyaguchi et al, Hepato-Gastroenterology 47 1518 (2000)).

However, there is no suggestion in the publications of Day et al., Nakai et al, Kurihara et al., or Miyaguchi et al. of the effectiveness of a combination of a bile acid and a fibrate in the treatment of hyperlipidaemia by lowering triglyceride and/or cholesterol levels in the blood. There is also no suggestion in these publications of the effectiveness of a combination of a bile acid and a fibrate for lowering elevated liver function tests associated with conditions other than primary biliary cirrhosis. Further, there is no suggestion in these publications of the effectiveness of a combination of a fibrate with either ursodiol sulfate or ursodiol bicarbonate for lowering elevated liver function tests.

In the compositions, methods and uses of the present invention, the bile acid may be a free bile acid or it may be in the form of a pharmaceutically acceptable salt. The bile acid is typically selected from the group consisting of chenodeoxycholic acid, ursodeoxycholic acid, ursodiol bicarbonate and ursodiol sulfate. In preferred compositions, methods and uses of the present invention, the bile acid is ursodeoxycholic acid. In other preferred compositions, methods and uses of the present invention, the bile acid is ursodiol bicarbonate. In further preferred compositions, methods and uses of the present invention, the bile acid is ursodiol sulfate.

In the compositions, methods and uses of the present invention, the fibrate is typically selected from the group consisting of bezafibrate, fenofibrate gemfibrozil, ciprofibrate, binifibrate, clinofibrate, clofibrate, clofibric acid, clofibride, etofibrate, nicofibrate, pirifibrate, plafibride, ronifibrate, theofibrate, tocofibrate and derivatives of 2-phenoxy-2-methylpropanoic acid in which the phenoxy moiety is substituted with an optionally substituted residue of piperidine, 4-hydroxypiperidine, piperid-3-ene or piperazine, as disclosed in European patent application no, 607536. An example of the latter group of substances is 2-[3-[1-(4-fluorobenzoyl)piperidin-4-yl]phenoxy-2-methyl-propanoic acid. In preferred compositions, methods and uses of the present invention, the fibrate is bezafibrate, fenofibrate, gemfibrozil or ciprofibrate. In more preferred compositions, methods and uses of the present invention, the fibrate is bezafibrate.

In a particularly preferred form of the compositions, methods and uses of the present invention, the fibrate is bezafibrate and the bile acid is ursodiol bicarbonate.

In one form of the invention, the bile acid aid fibrate are administered in a three-way combination with a statin, such as simvastatin, fluvastatin, pravastatin, atorvastatin, cerivastatin, or lovastatin. In his form of the invention, a bile acid, together with a fibrate and a statin can for the first time achieve profound, synergistic reductions in severe, combined hyperlipidaemic states and those resistant to individual therapies. Hence, for the very difficult to control hyperlipidaemias a combination of a fibrate, bile acid and a statin are advantageous. It is particularly advantageous for such a combination of a fibrate, a bile acid and a statin to be provided in a single capsule form designed to increase compliance and hence effectiveness.

Accordingly, the invention further provides a pharmaceutical composition comprising an effective amount of at least one bile acid, an effective amount of at least one fibrate and an effective amount of at least one statin, together with one or more pharmaceutically acceptable carriers, diluents, adjuvants or excipients,

The invention still further provides a method for the treatment of hyperlipidaemia in a patient in need of said treatment, comprising administering to said patient an effective amount of at least one bile acid, an effective amount of at least one fibrate and an effective amount of at least one statin.

A composition in accordance with the present invention will typically contain sufficient of the bile acid, the fibrate and, optionally, the statin to permit the desired daily dose of each to be administered to a patient in a single unit dosage form, such as a tablet or capsule, or in two or more unit dosage forms to be adminstered simultaneously or at intervals during a day.

The pharmaceutical compositions and methods of the present invention may be used for the treatment of all forms of hyperlipidaemias. For example, they may be used for the treatment of hyperlipidaemia where the hyperlipidaemia is pay hyperlipidaemia with or without a genetic component; or hyperlipidaemia associated with coronary artery disease, cerebrovascular arterial disease, peripheral vascular disease, aortic aneurisms and carotid atherosclerosis or where it is associated with a condition selected from resistant primary biliary cirrhosis; primary biliary cirrhosis where there is associated liver function test elevation and hyperlipidaemia; primary sclerosing cholangitis, non-alcohol-induced steatohepatosis; and chronic liver disease associated with hepatitis B, C or alcohol.

The pharmaceutical compositions and methods of the present invention also have an application in patients with Primary Sclerosing Cholangitis for similar biochemical abnormalities as well as in patients with chronic hepatitis caused by hepatitis D, C and by alcohol. Furthermore, they can also be used in other arterial disorders associated with hyperlipidaemia.

Another application of the methods of the present invention is in the treatment of non alcohol-induced steatohepatitis (NASH), where not only lipid profiles improve but also various liver function tests improve and fatty liver is progressively reversed. Ian et al (Hepatology 23 1464 (1996)) described the modest effect of UDCA in NASH but found bezafibrate to have no effect. This group did not study the effect of a combination of both drugs. In the present invention it was found that NASH liver function tests indeed respond markedly in most NASH patients to whom a combination of bile acid and fibrate are administered, showing unexpected synergy of bile acid and fibrate.

In the methods of the present invention the active substances may be administered in single daily doses, or in two, three, four or more identical or different divided doses per day, and they may be administered simultaneously or at different times during the day. Usually, the active substances will be administered simultaneously, more usually in a single combined dosage form.

In the methods of the present invention the bile acid(s), fibrate(s) and, optionally, statin(s) are typically administered in dosages substantially the same as the dosages in which they are administered in prior art monotherapies. For example, the daily dosages used in a typical hyperlipidaemia therapy of the present invention employing bezafibrate and a bile acid will usually include 10-1000 mg of bezafibrate, more usually about 200-500 mg, even more usually about 400 mg; and 10-2000 mg, more typically about 200-500 mg, even more typically about 250 mg of ursodeoxycholic acid, or 10-2000 mg ursodiol bicarbonate, more typically about 200-500 mg, or 10-2000 mg ursodiol sulfate, more typically about 200-500 mg. Daily dosages of other fibrates and bile acids for administration according to prior art regiments are known to persons of ordinary skill in the art. Where a statin is also included, from about 1-500 mg of the statin more typically about 10 mg, will be administered daily.

Compositions of the present invention therefore typically include from about 10-1000 mg of fibrate, more typically about 200 mg; from about 10-2000 mg of a bile acid, more typically about 200 mg; and optionally from about 1-500 mg of a statin, more typically about 10 mg.

A pharmaceutical composition of the present invention may be in any convenient form for oral administration, such as a tablet, capsule, powder, lozenge, pill, troche, elixir, lyophilised powder, solution, granule, suspension, emulsion, syrup or tincture. Slow-release, or delayed-release forms may also be prepared, for example in the form of coated particles, multi-layer tablets, capsules within capsules, tablets within capsules, or microgranules.

Solid forms for oral administration may contain pharmaceutically acceptable binders, sweeteners, disintegrating agents, diluents, flavourings, coating agents, preservatives, lubricants and/or tire delay agents. Suitable binders include gum acacia, gelatin, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers or acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further include dispersing agents and/or suspending agents Suitable suspending agents include sodium carboxymethylcellulose, methylcellulose, hydroxypropylmethylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further include one or more emulsifying agents, Suitable emulsifying agents include dispersing agents as exemplified above or natural gums such as gum acacia or gum tragacanth.

Pharmaceutical compositions of the present invention may be prepared by blending, grinding, homogenising, suspending, dissolving, emulsifying, dispersing and/or mixing the bile acid(s) and the fibrate(s), and optionally the statin(s) together with the selected excipient(s), carrier(s), adjuvant(s) and/or diluent(s). One type of pharmaceutical composition of the present invention in the form of a tablet or capsule may be prepared by (a) preparing a first tablet comprising at least one of the active substances selected from the bile acid(s) and the fibrate(s), together with any desired excipient(s), carrier(s), adjuvant(s) and/or diluent(s), and (b) preparing a second tablet or a capsule, wherein the second tablet or the capsule includes the remaining active substance(s) and the first tablet. Another type of pharmaceutical composition of the present invention in the form of a capsule may be prepared by (a) preparing a first capsule comprising at least one of the active substances selected from the bile acid(s) and the fibrate(s), together with any desired excipient(s), carrier(s), adjuvant(s) and/or diluent(s), and (b) preparing a second capsule, where the second capsule includes the remaining active substance(s) and the first tablet. A fiber type of pharmaceutical composition of the present invention in the form of a tablet may be prepared by (a) preparing a capsule comprising at least one of the active substances selected from the bile acid(s) and the fibrate(s), together with any desired excipient(s), carrier(s), adjuvant(s) and/or diluent(s), and (b) preparing a tablet, wherein the tablet includes the remaining active substance(s) and the capsule.

In preferred compositions, methods and uses of the invention the fibrate is used either as an immediate release tablet or as a sustained release tablet. It is particularly effective when provided in a sustained release tablet. Sustained release tablets of fibrates are commercially available. It is preferable for prolonged action that the tablet is in a sustained release format.

One preferred form of the compositions of the invention is a dosage form which comprises a sustained release tablet of bezafibrate, in an amount of 10-1000 mg, more typically about 200 mg, contained within a capsule which contains ursodeoxycholic acid in an amount of from 10 mg-2000 mg, more typically about 200 mg, In this way the patient to whom the dosage form is administered receives a sustained release tablet of bezafibrate which is delivered to the distal antrum as the capsule breaks open and releases ursodeoxycholic acid. Ursodeoxycholic acid is delivered simultaneously with the bezafibrate so achieving a profound suppression of lipids and at the same time reversing the elevation of hepatic transaminases and other liver function tests The combination of ursodeoxycholic acid and bezafibrate is a much more effective therapy than UDCA or bezafibrate alone for (for example) general patients with primary biliary cirrhosis and hyperlipidaemia, and especially for ‘UDCA-resistant’ primary biliary cirrhosis.

The methods of the present invention can be used lifelong by the patient, prolonging survival and delaying liver transplantation. At the same time the reduction of hyperlipidaemia ensures reduction in the development of associated vascular disease. Both bile acids and fibrates have very Hal long-term side effect profile (with some exceptions for bezafibrate) and therefore this combination is likely to be the therapy of choice for primary biliary cirrhosis with hyperlipidaemia and for resistant primary biliary cirrhosis. Because of the simplified dosing provided by the methods of the present invention, a combined therapy of the present invention can be used in increasing doses, depending on a patient's weight and clinical response.

Another preferred composition of the present invention comprises a capsule containing the fibrate within a capsule containing the bile acid. Typically in this form the fibrate is presented in an immediate release form. In that event it is usual to administer the composition three times daily. Another preferred mode of administration is to provide a composition containing the fibrate in either a sustained release or a non-sustained release form as described above, twice daily, wherein the daily amount of the composition administered contains sufficient of the active substances to give the desired daily dosage for the patient.

A composition of the present invention that comprises a bile acid, a fibrate and a statin is typically provided as the three active substances within a single capsule. In one form of such a composition, a statin may be mixed with a bile acid (which is chemically poorly reactive) in an inner capsule, the inner capsule being surrounded by a fibrate contained within an outer capsule. The locations within the capsules may be reversed. That is, the mixture of statin and bile acid may be contained within the outer capsule and the fibrate may be contained within the inner capsule. This arrangement will be especially desirable if the quantity of the statin to be administered is relatively large. Other combinations for presentation of the combination of three active substances are possible.

A combination of a bile acid, a fibrate and a statin can be used for all hyperlipidaemias, but is especially indicated in patients who have both cholesterol and triglycerides elevated. By including all three active substances in a single dosage form various mechanisms of hypercholesterolaemia and hypertriglyceridaemia are blocked, and the medications are delivered simultaneously so as to increase patients' compliance.

### EXAMPLES

### Example 1

### Treatment of Hyperlipidaemia Associated with Primary Biliary Cirrhosis

A 56 year old female patient with primary biliary cirrhosis and hypertriglyceridaemia had been treated with UDCA alone for several years. However, her condition although improving partially was resistant to 2 grams of ursodeoxycholic acid and her alkaline phosphatase, γ-glutamyl transpeptidase (GGT), aspartate aminotransferase (AST) and alanine aminotransferase (ALT) remained elevated. Slow release bezafibrate 400 mg was combined with 250 mg of UDCA and the combination was administered twice daily—a dose which would not have previously made any change in her liver function tests. After four weeks her alkaline phosphatase fell from 287 units to 95 (normal level) and continued to fall in the next four weeks to 69, Her GGT fell from 627 to 252 and foul weeks later to 157, Her AST fell from 48 to 25 and ultimately to 18-well below normal levels. Her ALT fell from 65 to 30 and ultimately also to 18.

In addition, her cholesterol was reduced from 7.9 to 6.4 (a fall of 18%) and her triglycerides from 2.4 to 0.6 (a fall of more than 70%). The patient also improved clinically feeling much more energetic for otherwise unexplainable reasons.

### Example 2

### Treatment of Hyperlipidaemia and Elevated Liver Function Tests Associated with NASH

A male of 38 years of age was diagnosed as having elevated liver function tests of unknown origin having had all other known causes excluded liver biopsy showed macrovesicular steatotosis, mild lobular inflammation and some liver cell degeneration. His ultrasound demonstrated hyper reflection consistent with liver biopsy of a fatty liver and the diagnosis of NASH was made. Although the patient reduced his weight modestly, the liver function test did not change. Treatment with a combined therapy using 250 mg twice daily of UDCA together with bezafibrate 400 mg sustained-release tablet Twice daily reduced by 50% his AST from 96 to <40 and ALT 110 to <50 at 8 weeks. His triglyceride level also fell from 2.1 to 1.7.

1.-9. (canceled)

10. A method of lowering elevated liver function tests in a patient comprising,
administering to a patient having elevated liver function tests associated with chronic hepatitis, an effective amount of at least one bile acid and an effective amount of at least one fibrate.

11. (canceled)

12. The method according to claim 10, wherein said bile acid is selected from the group consisting of chenodeoxycholic acid, ursodeoxycholic acid, ursodiol bicarbonate and ursodiol sulfate.

13. The method according to claim 10, wherein said fibrate is selected from the group consisting of bezafibrate, fenofibrate, gemfibrozil, ciprofibrate and derivatives of 2-phenoxy-2-methylpropanoic acid in which the phenoxy moiety is substituted with an optionally substituted residue of piperidine, 4-hydroxypiperidine, piperid-3-ene or piperazine.

14. The method according to claim 10, wherein said bile acid is ursodiol bicarbonate and said fibrate is bezafibrate.

15. The method according to claim 10 further comprising administering to said patient an effective amount of at least one statin.

16. The method according to claim 10, wherein one of said bile acid and said fibrate is contained within a first capsule, and the other of said bile acid and said fibrate is contained within a second capsule, said first capsule being contained within said second capsule.

17.-18. (canceled)

19. The method according to claim 15, wherein said statin is selected from a group consisting of simvastatin, fluvastatin, pravastatin, atorvastatin, cerivastatin, and lovastatin.

20. The method according to claim 15, wherein said statin is fluvastatin.

21. The method of claim 10, wherein said chronic hepatitis is associated with hepatitis B infection.

22. The method of claim 10, wherein said chronic hepatitis is associated with hepatitis C infection.

23. The method of claim 10, wherein said chronic hepatitis is associated with alcohol consumption.

24. A method of treating chronic hepatitis in a patient comprising,
administering to a patient having chronic hepatitis, an effective amount of at least one bile acid and an effective amount of at least one fibrate.

25. The method of claim 24, wherein said chronic hepatitis is associated with hepatitis B infection.

26. The method of claim 24, wherein said chronic hepatitis is associated with hepatitis C infection.

27. The method of claim 24, wherein said chronic hepatitis is associated with alcohol consumption.

28. The method of claim 24, wherein said bile acid is selected from the group consisting of chenodeoxycholic acid, ursodeoxycholic acid, ursodiol bicarbonate and ursodiol sulfate.

29. The method of claim 24, wherein said fibrate is selected from the group consisting of bezafibrate, fenofibrate, gemfibrozil, ciprofibrate and derivatives of 2-phenoxy-2-methylpropanoic acid in which the phenoxy moiety is substituted with an optionally substituted residue of piperidine, 4-hydroxypiperidine, piperid-3-ene or piperazine.

30. The method of claim 24, wherein said bile acid is ursodiol bicarbonate and said fibrate is bezafibrate.

31. The method of claim 24 further comprising administering to said patient an effective amount of at least one statin.

32. The method according to claim 31, wherein said statin is fluvastatin.

33. The method of claim 24, wherein one of said bile acid and said fibrate is contained within a first capsule, and the other of said bile acid and said fibrate is contained within a second capsule, said first capsule being contained within said second capsule.

34. A method of treating chronic hepatitis in a patient comprising,
administering to a patient having chronic hepatitis, an effective amount of at least one bile acid, an effective amount of at least one fibrate and an effective amount of at least one statin, wherein said statin is selected from a group consisting of simvastatin, fluvastatin, pravastatin, atorvastatin, cerivastatin, and lovastatin.

35. A method of treating non-alcohol-induced steatohepatitis (NASH) in a patient comprising,
administering to a patient having NASH, an effective amount of at least one bile acid and an effective amount of at least one fibrate.
